(19) United States
(12) Patent Application Publication
(10) Pub. No.: US 2001/0049128 A1
(43) Pub. Date: Dec. 6, 2001

(54) PROCESS FOR THE FERMENTATIVE PRODUCTION OF L-AMINO ACIDS

(76) Inventors: ULRICH BECKER, SELCE (DE); HEIDI PETER, LEICHLINGEN (DE); SUSANNE MORBACH, JULICH (DE); ILONA WALGER, BIELEFELD (DE); REINHARD KRAMER, JULICH (DE); WALTER PFEFFERLE, HALLE (DE)

Correspondence Address: SMITH GAMBRELL & RUSSELL LLP, BEVERIDGE DEGRANDI WEILACHER & YOUNG, INTELLECTUAL PROPERTY GROUP, 1850 M STREET NW SUITE 800, WASHINGTON, DC, 20036

(21) Appl. No.: 09/428,048

(22) Filed: Oct. 27, 1999

(30) Foreign Application Priority Data
Oct. 28, 1998 (DE) 198 49 625.7

(51) Int. Cl. C12P 13/14; C12P 13/04

(52) U.S. Cl. 435/110; 435/106

(57) ABSTRACT

A process for the fermentative production of L-amino acids using bacteria, wherein L-proline is added to the fermentation broth as an osmoprotective substance in order to suppress the effects on the cells of the hyperosmotic stress.

### INTRODUCTION AND BACKGROUND

The present invention relates to a process for the fermentative production of L-amino acids using coryneform bacteria, wherein L-proline is added to the fermentation broth as an osmoprotective substance.

It is known that, under osmotic stress, most microorganisms concentrate potassium ions or so-called osmolytes (organic compounds) in their cytoplasm. This leads to an internal osmotic resistance, which prevents the dehydration of the cells. In this connection, it is known that the addition of glycine betaine stimulates the growth rate of the cells, particularly in media with inhibiting osmotic stress. This leads to a rise in the rate of sugar consumption and to an increase in the production of L-lysine (Y. Kawahara, Y. Yoshihara, S. Ikeda, H. Yoshii, Y. Hirose, Stimulatory effect of glycine betaine on L-lysine fermentation (1990), 34 (1), pp 87-90, Applied Microbiology Biotechnology).

In the case of proline-auxotrophic mutants of Brevibacterium lactofermentum, it has been found that proline plays a part in osmoregulation.

The osmotic tolerance of these strains has proved to be lower than that of the wild strain.

In this connection, the activity of the pyrroline-5-carboxylate reductase is found to have increased three times when the cells grew under osmotic stress (Y. Kawahara, T. Ohsumi, Y. Yoshihara, S. Ikeda, Proline in the Osmoregulation of Brevibacterium lactofermentum, (1989), 53, (9), pp 2475-2479, Agricultural and Biological Chemistry).

The production of amino acids is not to be found in the reference cited.

It is an object of the present invention to enable the fermentative production of L-amino acids, wherein the effects on the cells of the hyperosmotic stress are suppressed.

### SUMMARY OF THE INVENTION

The above and other objects of the present invention can be achieved by a process for the fermentative production of L-amino acids, comprising cultivating Coryneform and other microorganisms sensitive to hyperosmotic stress suppression which produce and excrete L-amino acids in a medium to which, besides the conventional constituents, L-proline or L-proline derivatives are added, preferably at the beginning of the fermentation. It is applicable in particular to so-called minimal media and defined media, which consist of constituents identified by quantity and type. But the addition of L-proline or its derivative also results in improved yields in the case of complex media, the contents of which include hydrolysates or extracts.

In this process L-proline or its derivative does not serve as a source of carbon or of nitrogen in the metabolism of the microorganisms. But the addition brings about the improved growth of the amino acid producers and an increase in the yield of L-amino acid.

The invention may also be practiced with any strain of microorganism containing pyroline-5-carboxylate reductase, so long as that microorganism is also osmotically sensitive to the presence of L-proline or its derivative. The reductase may be naturally produced by the microorganism, or it may be produced using recombinant methods.

The detention and analysis of the suppressed hyperosmotic stress induced by the presence of L-proline, or its derivative, in the medium, can be conventionally practiced by any method known in the art. The detection of this hyperosmotic stress suppression sensitivity, in combination with the presence of the pyrroline-5-carboxylate reductase in the microorganism can be used to identify candidate microorganisms to be used in the method of the invention for enhanced production of L-amino acids.

### DETAILED DESCRIPTION OF THE INVENTION

Coryneform microorganisms, in particular the species Corynebacterium glutamicum, have long been known as amino-acid producers. Preferably strains which are suitable for the production of L-lysine, L-isoleucine, L-threonine or L-valine are used. L-glutamic acid can also be produced in this way.

The fermentation is generally carried out at temperatures between 25° C. and 50° C., preferably at 30° C. to 45° C., while the pH is between 6 and 8, preferably 7 and 7.5, and the ammonium concentration is preferably between 0.5 and 8 g/l.

L-proline is added to the fermentation broth in a quantity of between 0.01 and 10 g/l, preferably between 0.1 and 2.5 g/l. L-proline derivatives may substitute for L-proline. L-proline derivatives according to the invention are any chemical variant of L-proline with substitents that do not destroy the osmotic stress suppression sensitivity that manifests itself in conjunction with the presence of the L-proline derivative in the medium with the mircroorganism.

Such substituents include, but are not limited to: alkyl, alkoxy, haloalkoxy, phenyl, aryl, or aralkyl. These substituents may include functionalities that can be, but are not limited to: alcohols, phenols, ethers, epoxides, acrylates, glycols, aldehydes, ketones, carboxylic acids, or anhydrides and amines. Other substituents may be amino acids, polypeptides, proteins, synthetic polymers, lipids and carbohydrates or others; so long as the osmotic stress suppression sensitivity of the mircroorganism to the L-proline derivative is not destroyed.

Suitable strains of the genus Corynebacterium, in particular the species Corynebacterium glutamicum, are, for example, the known wild strains which produce glutamic acid:

Corynebacterium glutamicum ATCC13032

Corynebacterium acetoglutamicum ATCC15806

Corynebacterium acetoacidophilum ATCC13870

Brevibacterium flavum ATCC14067

Brevibacterium lactofermentum ATCC13869 and

Brevibacterium divaricatum ATCC14020

and mutants or strains produced therefrom, such as, for example, the L-lysine-producing strains

Corynebacterium glutamicum FERM-P 1709

Brevibacterium flavum FERM-P 1708 and

Brevibacterium lactofermentum FERM-P 1712

or such as, for example, the L-threonine-producing strains

Corynebacterium glutamicum FERM-P 5835

Brevibacterium flavum FERM-P 4164 and

Brevibacterium lactofermentum FERM-P 4180

or such as, for example, the L-isoleucine-producing strains

Corynebacterium glutamicum FERM-P 756

Brevibacterium flavum FERM-P 759 and

Brevibacterium lactofermentum FERM-P 4192

or such as, for example, the L-valine-producing strains

Brevibacterium flavum FERM-P 512 and

Brevibacterium lactofermentum FERM-P 1845.

The media used for the fermentation are known basal media for the production of L-amino acids which are mentioned in the present invention, or media that are conventionally used for the production of L-amino acids and are suitable for bacteria which produce L-amino acids.

The main sources of carbon used, as is generally known, are sugars, such as glucose, saccharose, fructose, maltose, molasses, also starch and starch hydrolysate, cellulose and saccharified cellulose, lactose; fatty acids, such as acetic acid, propionic acid, palmitic acid, stearic acid, linoleic acid; organic acids, such as pyruvic acid, citric acid, succinic acid, fumaric acid, malic acid; alcohols, such as ethyl alcohol, butyl alcohol; individual components or mixtures of the above-mentioned compounds. In addition, precursors from the biosynthetic pathway of the chosen L-amino acid and the latter itself can be used.

The source of phosphorus used is generally phosphoric acid, potassium dihydrogen phosphate or dipotassium hydrogen phosphate or the corresponding sodium-containing salts.

Sources of nitrogen used, as is generally known, are ammonium salts, such as ammonium sulfate, ammonium chloride, ammonium nitrate, ammonium acetate, urea, liquid ammonium or ammonia water. Complex organic sources of nitrogen used are casamino acids, maize steep liquor, soya flour hydrolysate, yeast extract, biomass hydrolysates and protein hydrolysates.

Inorganic salts which can be used are phosphates, magnesium salts, calcium salts, potassium salts, sodium salts, iron salts, manganese salts, zinc salts, copper salts and other trace elements, if necessary. In addition, if necessary, vitamins such as biotin, thiamine, and others, can be used.

The cultivation conditions according to the present invention are the same as in the known amino acid fermentations. Whereas the compositions of the fermentation broths vary, depending upon the L-amino acid or the strain used, the cultivation temperature is 25° C. to 50° C., preferably 30° C. to 45° C. With regard to the pH value, good results are obtained when the pH value remains within the neutral range. Where protein hydrolysate is used as a complex source of nitrogen, the proline content which may be present therein is advantageously taken into account in the calculation of the additional proline used. The quantity of proline originating from the hydrolysate is limited by the natural composition of these products, so that the addition of further quantities of proline within the framework of the process according to the invention proves to be advantageous.

### EXAMPLES

The present invention is explained in more detail below by means of Examples.

To this end, tests with amino acid-producing strains were carried out, in which the superiority of the claimed process is demonstrated:

a) the L-lysine-producing strain Corynebacterium glutamicum DSM5715, (EP-B 0 435 132) and

b) the L-threonine- and L-isoleucine-producing strain Brevibacterium flavum DSM5399 (EP-B 0 385 940).

### Example 1

Fermentative Production of L-lysine

A culture medium containing 2.5 g/l NaCl, 10 g/l peptone and 10 g/l yeast extract was adjusted to pH 7.4 with sodium hydroxide and, after heat sterilisation, 40 ml of 50% glucose solution per liter was added thereto. 47 ml portions of the medium were inoculated with Corynebacterium glutamicum DSM5715 with a needle on an agar plate with brain-heart agar as nutrient medium incubated for 48 hours and were shaken at 150 rpm for 20 hours at 33° C. in an RC-1-TK incubator from the firm Infors AG (Bottmingen, Switzerland). The cells were then washed with sterile physiological saline. The cells were separated by centrifugation for 20 minutes at 4000 rpm in a Beckmann centrifuge J 6B.

For the main cultivation in shaking flasks, 40 g (NH₄)₂SO₄, 0.5 g KH₂PO₄, 0.5 g K₂HPO₄, 0.25 g MgSO₄.7H₂O and 0.3 g L-leucine were weighed in a 1 l beaker and 750 ml distilled water was added thereto. 1 ml of a solution of trace salts was also added. The solution of trace salts contained 1.0 g FeSO₄.7H₂O, 1.0 g MnSO₄.H₂O, 0.1 g ZnSO₄.7H₂O, 0.02 g CUSO₄ and 0.002 g NiCl₂.6H₂O, which were dissolved in 100 ml distilled H₂O, slightly acidified with a few drops of HCl in order to increase the solubility of the salts. In addition, 1 ml of a solution of 0.02 g biotin per 100 ml distilled H₂O was added. Then NaCl was added in a concentration of 5 g/l. This cultivation medium was divided into 45 ml portions, which were placed in 500 ml Erlenmeyer flasks and adjusted to different concentrations of proline, ranging from 0.1 to 10 g/l. After a heat sterilisation in an autoclave at 121° C. for 20 minutes, 12 ml of a separately sterilised 50% glucose solution and 1.2 g sterilised CaCO₃ were added to each flask. Inoculation then took place with the cells of the culture medium, which had been washed under sterile conditions. The optical density (wavelength used in determination: 535 nm) of the washed cells was 18.5; 7.7 ml of this suspension was used for the inoculation of 57 ml of culture medium.

The cultivation took place over 72 hours at 33° C. and 150 rpm in an RC-1-TK incubator from the firm Infors AG (Bottmingen, Switzerland). Subsequent to this, the optical density (OD) (photometer LP2W from the firm Dr. Lange, Berlin, Germany) and the concentration of L-amino acid formed in the culture suspension were determined. Amino acids were analysed by ion-exchange chromatography and post-column reaction with ninhydrin detection, using an amino acid analyser from the firm Eppendorf BioTronik (Hamburg, Germany). The result of the test is shown in Table 1.

TABLE 1

| Proline [g/l] | OD 535 nm | Lysine [g/l] |
|---|---|---|
| 0 | 24.6 | 23.6 |
| 0.5 | 30.5 | 29.4 |

### Example 2

Fermentative Production of L-threonine

A culture medium containing 100 g/l saccharose, 12 g/l (NH₄)₂SO₄, 100 ml/l soya flour hydrolysate, 0.5 g/l K₂HPO₄, 0.5 g/l KH₂PO₄, 0.25 g/l MgSO₄.7H₂O, 5.0 g/l NaCl and 1 ml of a solution of trace salts was adjusted to pH 7.0 and autoclaved. The solution of trace salts consisted of 1.0 g FeSO₄.7H₂O, 1.0 g MnSO₄.H₂O, 0.1 g ZnSO₄.7H₂O, 0.02 g CuSO₄ and 0.002 g NiCl₂.6H₂O, which was made up to 100 ml with demineralised water and a few drops of a 1N HCl solution.

1 ml each of a 0.2 mg/l biotin and thiamine stock solution, which had been sterilised by filtration, were added to the culture medium. 10.0 g/l CaCO₃ was sterilised together with the shaking flasks. In the culture medium, the proline concentration resulting from the introduction of soya flour hydrolysate was 0.34 g/l. The specified concentration of proline, obtained from a proline stock solution, was added to the medium after having been sterilised by filtration.

An agar plate with brain-heart agar as nutrient medium, which had been incubated for 72 hours with DSM5399, was suspended in 10 ml of sterile physiological saline. 10 ml portions of cultivation medium were placed in 100 ml Erlenmeyer shaking flasks and inoculated with 100 μl of the withdrawn cell suspension. The cultivation took place over 72 hours at 30° C. and 300 rpm. Subsequent to this, as specified in Example 1, the OD was determined at a wavelength of 660 nm and the threonine concentration was measured. The result of the test is shown in Table 2.

TABLE 2

| Proline [g/l] | OD 660 nm | Threonine [g/l] |
|---|---|---|
| 0.34 | 51.2 | 0.63 |
| 0.66 | 52.6 | 1.29 |

### Example 3

Fermentative Production of L-isoleucine

A culture medium containing 100 g/l saccharose, 12 g/l (NH₄)₂SO₄, 0.5 g/l K₂HPO₄, 0.5 g/l KH₂PO₄, 0.25 g/l MgSO₄.7H₂O, 5.0 g/l NaCl and 1 ml of a solution of trace salts was adjusted to pH 7.0 and autoclaved. The solution of trace salts consisted of 1.0 g FeSO₄.7H₂O, 1.0 g MnSO₄.H₂O, 0.1 g ZnSO₄.7H₂O, 0.02 g CuSO₄ and 0.002 g NiCl₂.6H₂O, which was made up to 100 ml with demineralised water and a few drops of a 1N HCl solution.

1 ml each of a 0.2 mg/l biotin and thiamine stock solution, which had been sterilised by filtration, were added to the culture medium. 10.0 g/l CaCO₃ was sterilised together with the shaking flasks. The appropriate concentration of praline, obtained from a praline stock solution, was added to the culture medium after having been sterilised by filtration.

An agar plate with brain-heart agar as nutrient medium, which had been incubated for 72 hours with DSM5399, was suspended in 10 ml sterile physiological saline. 10 ml portions of cultivation medium were placed in 100 ml Erlenmeyer shaking flasks and inoculated with 100 μl of the withdrawn cell suspension. The cultivation took place over 72 hours at 30° C. and 300 rpm. Subsequent to this, as specified in Example 1, the OD was determined at a wavelength of 660 nm and the isoleucine concentration was measured. The result of the test is shown in Table 3.

TABLE 3

| Proline [g/l] | OD 660 nm | L-isoleucine [g/l] |
|---|---|---|
| 0 | 51.2 | 0.18 |
| 0.1 | 52.0 | 0.36 |

Further variations and modification of the foregoing will be apparent to those skilled in the art and are intended to be encompassed by the claims appended hereto.

German application 198 49 625.7 is relied on and incorporated herein by reference.

We claim:

1. A process for the fermentative production of L-amino acids comprising cultivating a microorganism which produces and excretes an L-amino acid, by adding L-proline, or L-proline derivative, to the fermentation broth containing a source of carbon and nitrogen.

2. The process according to claim 1, wherein the L-proline, or L-proline derivative, is added at the beginning of fermination.

3. The process according to claim 1, wherein L-proline, or L-proline derivative, is added in a quantity of from 0.01 to 10 g/l, based on the fermentation broth.

4. The process according to claim 2, wherein L-proline or L-proline derivative is added in a quantity of from 0.1 to 2.5 g/l, based on the fermentation broth.

5. The process according to claim 1, wherein the fermentation is carried out in a minimal medium and/or defined medium.

6. The process according to claim 2, wherein the fermentation is carried out in a minimal medium and/or defined medium.

7. The process according to claim 3, wherein the fermentation is carried out in a minimal medium and/or defined medium.

8. The process according to claim 4, wherein the fermentation is carried out in a minimal medium and/or defined medium.

9. The process according to claim 1, wherein fermentation is carried out in a medium containing hydrolysate.

10. The process according to claim 2, wherein fermentation is carried out in a medium containing hydrolysate.

11. The process according to claim 3, wherein fermentation is carried out in a medium containing hydrolysate.

12. The process according to claim 4, wherein fermentation is carried out in a medium containing hydrolysate.

13. The process according to claim 1, wherein L-glutamic acid, L-lysine, L-isoleucine, L-threonine or L-valine are produced.

14. The process according to claim 1, wherein said microorganism is of the genus Corynebacterium.

15. A process according to claim 1, wherein said microorganism contains recombinantly produced pyrroline-5-carboxylate reductase.

16. A process according to claim 1, wherein said microorganism is of the genus Brevibacterium.

17. A process according to claim 1, wherein said microorganism is an L-glutamic acid producing microorganism.

18. A process according to claim 1, wherein said microorganism is an L-lysine acid producing microorganism.

19. A process according to claim 1, wherein said microorganism is an L-threonine acid producing microorganism.

20. A process according to claim 1, wherein said microorganism is an L-isoleucine acid producing microorganism.

21. A process according to claim 1, wherein said microorganism is an L-valine acid producing microorganism.
